(19) United States
(12) Patent Application Publication
(10) Pub. No.: US 2024/0238196 A1
(43) Pub. Date: Jul. 18, 2024

(54) FAST DISSOLVING ORAL FILM PREPARATION COMPRISING RIVAROXABAN

(71) Applicant: Shilpa Medicare Limited, RAICHUR (IN)

(72) Inventors: SUDHIR VASANTRAO GAMBHIRE, Bengaluru (IN); RITESH VINOD BIRLA, Bengaluru (IN); TOPPALADODDI KRISHNAMURTHY, Bengaluru (IN)

(73) Assignee: Shilpa Medicare Limited, RAICHUR (IN)

(21) Appl. No.: 18/563,876

(22) PCT Filed: Jun. 22, 2022
(86) PCT No.: PCT/IB2022/055239

§ 371 Date: Nov. 27, 2023

(30) Foreign Application Priority Data
Jun. 10, 2021 (IN) 202141025776

(51) Int. Cl. A61K 9/00; A61K 31/5377; A61K 47/10; A61K 47/12; A61K 47/20; A61K 47/32; A61K 47/36; A61K 47/38; A61K 47/44

(52) U.S. Cl. CPC A61K 9/0056; A61K 31/5377; A61K 47/10; A61K 47/12; A61K 47/20; A61K 47/32; A61K 47/36; A61K 47/38; A61K 47/44

(57) ABSTRACT

The present invention relates to an orally fast dissolving film formulation comprising rivaroxaban or pharmaceutically acceptable salts thereof and the process for preparation thereof. The present invention further relates to the fast dissolving film formulation comprising rivaroxaban or a pharmaceutically acceptable salt thereof and at least one solubilizer, wherein the film has high dissolution rate, causes no risk of damage to oral tissues and gives a good feeling, when placed on the tongue.

### FIELD OF THE INVENTION

The present invention relates to an orally fast dissolving film formulation comprising rivaroxaban. The orally fast dissolving film formulation includes rivaroxaban or a pharmaceutically acceptable salt thereof and at least one solubilizer. The orally fast dissolving film formulation has high dissolution rate, causes no risk of damage to oral tissues and gives a good feeling, when placed on the tongue.

### BACKGROUND OF THE INVENTION

Rivaroxaban a factor Xa inhibitor, is indicated to reduce the risk of stroke and systemic embolism in patients with nonvalvular atrial fibrillation, for treatment of deep vein thrombosis (DVT), for treatment of pulmonary embolism (PE), for prophylaxis of venous thromboembolism (VTE) and to reduce the risk of major cardiovascular events in patients with chronic coronary artery disease (CAD) or peripheral artery disease (PAD). The chemical name of rivaroxaban is 5-Chloro-N-({(5S)-2-oxo-3-[4-(3-oxo-4-morpholinyl)phenyl]-1,3-oxazolidin5-yl}methyl)-2-thiophenecarboxamide and it has the following structure

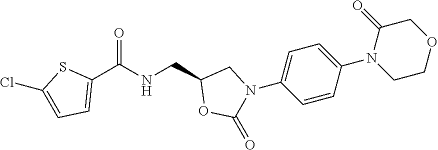 [SMILES: O=C(NC[C@H]1CN(c2ccc(N3CCOCC3=O)cc2)C(=O)O1)c1ccc(Cl)s1]

Rivaroxaban is marketed as conventional immediate release tablets comprising 2.5 mg, 10 mg, 15 mg and 20 mg of rivaroxaban with the brand name Xarelto®.

Rivaroxaban is practically insoluble in water and it has solubility of 7 mg/mL and has high permeability, thus being a BCS Class II Compound. According to CHMP assessment report for Xarelto®, rivaroxaban is slightly soluble in organic solvents (1.0-10.0 g/L).

The form of preparing and dispensing the conventional immediate release tablets of rivaroxaban has many disadvantages that includes a large proportion of adjuvants are added to the tablet dosage forms and further needs the additional storage space. In addition, large number of patients has difficulties in swallowing the conventional immediate release tablets of rivaroxaban.

As an alternate to the immediate release tablets orally fast dissolving film formulations of rivaroxaban are used to overcome the above disadvantages of difficulties in swallowing the conventional immediate release tablets of rivaroxaban.

PCT Publication No. WO 2020/030991 discloses a film pharmaceutical composition for application to the oral mucosa comprising a water-soluble film matrix comprising an active ingredient rivaroxaban, wherein the water-soluble film matrix is a polymeric mixture of polyvinyl pyrrolidone, polymeric alginate and/or pullulan. The film pharmaceutical composition as disclosed in WO '991 publication further contains absorption enhancers and non-ionic surfactants. The films as disclosed in WO '991 publication contains alginate polymer which has low chemical stability and has high sensitivity to degradation process. Further these films are brittle and have low flexibility which are difficult to handle and pack which are consumer friendly.

In order to overcome the above disadvantages like low solubility of rivaroxaban and difficulties in administration of the conventional tablet dosage forms, there exists a need to develop the orally fast dissolving film formulation comprising rivaroxaban that are chemically stable and flexible for handling which rapidly disintegrate in less than about 1 minute, when placed on the tongue and a film formulation which is bioequivalent to Xarelto®.

### SUMMARY OF THE INVENTION

In one embodiment the present invention provides a stable orally fast dissolving film formulation comprising rivaroxaban or pharmaceutically acceptable salts thereof.

In further embodiment the present invention provides an orally fast dissolving film formulation comprising rivaroxaban and at least one solubilizer.

In a further embodiment the present invention provides an orally fast dissolving film formulation comprising
(a) rivaroxaban and
(b) at least one solubilizer selected from group consisting of sodium lauryl sulfate and polyoxyl 40 hydrogenated castor oil.

In further embodiment the present invention provides an orally fast dissolving film formulation comprising
(a) rivaroxaban;
(b) at least one solubilizer selected from group consisting of sodium lauryl sulfate and polyoxyl 40 hydrogenated castor oil and
(c) a film forming polymer selected from group consisting of polyvinyl pyrrolidone, hydroxypropyl methylcellulose and pullulan.

In a further specific embodiment, the present invention provides an orally fast dissolving film formulation comprising
(a) rivaroxaban;
(b) a solubilizer selected from group consisting of sodium lauryl sulfate and polyoxyl 40 hydrogenated castor oil and
(c) a film forming polymer selected from group consisting of polyvinyl pyrrolidone and hydroxypropyl methylcellulose.

In a further specific embodiment, the present invention provides an orally fast dissolving film formulation comprising
(a) about 2% w/w to about 25% w/w rivaroxaban;
(b) about 0.1% w/w to about 5% w/w sodium lauryl sulfate and
(c) about 0.01% w/w to about 1% w/w polyoxyl 40 hydrogenated castor oil.

In another embodiment, the present invention provides an orally fast dissolving film formulation comprising
(a) about 2% w/w to about 25% w/w rivaroxaban;
(b) about 0.25% w/w to about 1% w/w sodium lauryl sulfate and
(c) about 0.01% w/w to about 1% w/w polyoxyl 40 hydrogenated castor oil.

In a further more specific embodiment, the present invention provides an orally fast dissolving film formulation comprising
(a) about 2% w/w to about 25% w/w rivaroxaban;
(b) about 0.1% w/w to about 5% w/w sodium lauryl sulfate;
(c) about 0.01% w/w to about 1% w/w polyoxyl 40 hydrogenated castor oil and
(d) a film forming polymer selected from group consisting of polyvinyl pyrrolidone, hydroxypropyl methylcellulose and pullulan.

In a further embodiment, the present invention provides an orally fast dissolving film formulation comprising
(a) about 2% w/w to about 25% w/w rivaroxaban;
(b) about 0.1% w/w to about 5% w/w sodium lauryl sulfate;
(c) about 0.01% w/w to about 1% w/w polyoxyl 40 hydrogenated castor oil and
(d) a film forming polymer selected from group consisting of polyvinyl pyrrolidone and hydroxypropyl methylcellulose.

In a further embodiment, the present invention provides an orally fast dissolving film formulation comprising
(a) rivaroxaban;
(b) at least one solublizer selected from the group consisting of sodium lauryl sulfate and polyoxyl 40 hydrogenated castor oil;
(c) at least one film forming polymer selected from the group consisting of polyvinyl pyrrolidone, hydroxypropyl methylcellulose and pullulan;
(d) at least one diluent selected from group consisting of lactose monohydrate microcrystalline cellulose and maltodextrin;
(e) at least one plasticizer selected from group consisting of triacetin, triethyl citrate, glycerol, polyethylene glycol and propylene glycol and
(f) at least one excipient selected from group of sweetening agents, flavouring agents and colouring agents.

In a further embodiment of the invention, the present invention provides an orally dissolving film formulation used for the treatment of stroke, systemic embolism in patients with nonvalvular atrial fibrillation, deep vein thrombosis (DVT), pulmonary embolism (PE), venous thromboembolism (VTE), chronic coronary artery disease (CAD) and peripheral artery disease (PAD).

### DETAILED DESCRIPTION OF THE INVENTION

The present invention relates to an orally fast dissolving film formulation comprising rivaroxaban or pharmaceutically acceptable salts thereof.

In one embodiment of the invention, the invention provides an orally fast dissolving film formulation comprising of about 1% w/w to about 50% w/w rivaroxaban, preferably about 2% w/w to about 25% w/w rivaroxaban, more preferably about 5% w/w to about 40% w/w rivaroxaban, even more preferably about 10% w/w to about 25% w/w rivaroxaban and most preferably about 15% w/w to about 20% w/w rivaroxaban based on the total weight of the film formulation.

In another embodiment of the invention, the invention provides an orally fast dissolving film formulation comprising rivaroxaban and a solubilizer.

The solubilizers used in the present invention are selected from the group consisting of sodium lauryl sulfate and polyoxyl 40 hydrogenated castor oil and/or combinations thereof. The film formulation of the present invention contains about 0.01% w/w to about 10% w/w solubilizer, preferably about 0.05% w/w to about 8% w/w solubilizer and more preferably about 0.1% w/w to about 5% w/w solubilizer based on the total weight of the film formulation.

In a further embodiment, orally fast dissolving film formulation of present invention contains the solubilizer, which is a mixture of sodium lauryl sulfate and polyoxyl 40 hydrogenate castor oil (Kolliphor RH 40).

In more preferred embodiment, an orally fast dissolving film formulation of the present invention contains about 0.01% w/w to about 8% w/w sodium lauryl sulfate, preferably about 0.05% w/w to about 6% w/w sodium lauryl sulfate, more preferably about 0.1% w/w to about 5% w/w of sodium lauryl sulfate and most preferably about 0.25% w/w to about 3% w/w of sodium lauryl sulfate based on the total weight of the film formulation.

In a preferred embodiment, an orally fast dissolving film formulation of the present invention contains about 0.001% w/w to about 5% w/w polyoxyl 40 hydrogenated castor oil, preferably about 0.005% w/w to about 4% w/w polyoxyl 40 hydrogenated castor oil and more preferably about 0.01% w/w to about 1% w/w polyoxyl 40 hydrogenated castor oil based on the total weight of the film formulation.

In a further embodiment, the present invention provides an orally fast dissolving film formulation comprising
(a) about 2% w/w to about 25% w/w rivaroxaban;
(b) about 0.1% w/w to about 5% w/w sodium lauryl sulfate and
(c) about 0.01% w/w to about 1% w/w polyoxyl 40 hydrogenated castor oil.

In a further embodiment, the present invention provides an orally fast dissolving film formulation comprising
(a) about 2% w/w to about 25% w/w rivaroxaban;
(b) about 0.25% w/w to about 3% w/w sodium lauryl sulfate and
(c) about 0.01% w/w to about 1% w/w polyoxyl 40 hydrogenated castor oil.

In a further embodiment, orally fast dissolving film formulation of the present invention contains a film forming polymer. The film forming polymer used in the present invention is selected from the group consisting of polyvinyl pyrrolidone, hydroxypropyl methylcellulose and pullulan. The most preferably used polymer in the present invention is a polymer mixture of polyvinyl pyrrolidone and hydroxypropyl methylcellulose.

In another embodiment, the present invention provides an orally fast dissolving film formulation comprising
(a) rivaroxaban;
(b) at least one solubilizer selected from group consisting of sodium lauryl sulfate and polyoxyl 40 hydrogenated castor oil and
(c) a film forming polymer selected from group consisting of polyvinyl pyrrolidone, hydroxypropyl methylcellulose and pullulan.

In a further embodiment, the present invention provides an orally fast dissolving film formulation comprising
(a) rivaroxaban;
(b) a solubilizer selected from group consisting of sodium lauryl sulfate and polyoxyl 40 hydrogenated castor oil and
(c) a film forming polymer selected from group consisting of polyvinyl pyrrolidone and hydroxypropyl methylcellulose.

In another embodiment, an orally fast dissolving film formulation of present invention contains about 1% w/w to about 40% w/w polyvinyl pyrrolidone, preferably about 2% w/w to about 35% w/w polyvinyl pyrrolidone, more preferably about 5% w/w to about 30% w/w polyvinyl pyrrolidone, even more preferably about 6% w/w to about 25% w/w polyvinyl pyrrolidone and most preferably about 10% w/w to about 20% w/w polyvinyl pyrrolidone based on the total weight of the film formulation.

In a further embodiment, an orally fast dissolving film formulation of present invention contains about 5% w/w to about 60% w/w hydroxypropyl methylcellulose, preferably about 10% w/w to about 55% w/w hydroxypropyl methylcellulose, more preferably about 15% w/w to about 50% w/w hydroxypropyl methylcellulose, even more preferably about 20% w/w to about 45% w/w hydroxypropyl methylcellulose and most preferably about 25% w/w to about 40% w/w hydroxypropyl methylcellulose based on the total weight of the film formulation.

In another embodiment, the present invention provides an orally fast dissolving film formulation comprising
(a) about 2% w/w to about 25% w/w rivaroxaban;
(b) about 0.25% w/w to about 3% w/w sodium lauryl sulfate;
(c) about 0.01% w/w to about 1% w/w polyoxyl 40 hydrogenated castor oil;
(d) about 10% w/w to about 20% w/w polyvinyl pyrrolidone; and
(e) about 25% w/w to about 40% w/w hydroxypropyl methylcellulose based on the total weight of the film formulation.

In another embodiment of the invention, the present invention comprises at least one diluent. Diluents used in the present invention are selected from the group consisting of lactose monohydrate, microcrystalline cellulose and maltodextrin. In one specific embodiment the diluent used in the present invention is a mixture of microcrystalline cellulose and maltodextrin.

In a further embodiment of the invention, the present provides an orally fast dissolving film formulation comprising
(a) rivaroxaban;
(b) a solubilizer selected from group consisting of sodium lauryl sulfate and polyoxyl 40 hydrogenated castor oil;
(c) a film forming polymer selected from group consisting of polyvinyl pyrrolidone and hydroxypropyl methylcellulose; and
(d) a diluent selected from group consisting of microcrystalline cellulose and maltodextrin.

In another embodiment of the invention, an orally fast dissolving film formulation of the present invention comprises about 0.5% w/w to about 50% w/w microcrystalline cellulose, preferably about 1% w/w to about 40% w/w microcrystalline cellulose, more preferably about 2% w/w to about 30% w/w microcrystalline cellulose and most preferably about 3% w/w to about 25% w/w microcrystalline cellulose based on the total weight of the film formulation.

In a further embodiment of the invention, an orally fast dissolving film formulation of the present invention comprises about 0.5% w/w to about 20% w/w maltodextrin, preferably about 1% w/w to about 15% w/w maltodextrin, more preferably about 2% w/w to about 13% w/w maltodextrin and most preferably about 3% w/w to about 10% w/w maltodextrin based on the total weight of the film formulation.

In another embodiment of the invention, the present invention provides an orally fast dissolving film formulation comprising
(a) about 2% w/w to about 25% w/w rivaroxaban;
(b) about 0.25% w/w to about 3% w/w sodium lauryl sulfate;
(c) about 0.01% w/w to about 1% w/w polyoxyl 40 hydrogenated castor oil;
(d) about 10% w/w to about 20% w/w polyvinyl pyrrolidone;
(e) about 25% w/w to about 40% w/w hydroxypropyl methylcellulose;
(f) about 3% w/w to about 25% w/w microcrystalline cellulose; and
(g) about 3% w/w to about 10% w/w maltodextrin; based on total weight of the film formulation.

In a further embodiment of the invention, the present invention comprises at least one plasticizer. Plasticizers used in the present invention are selected from the group consisting of triacetin, triethyl citrate, glycerol, polyethylene glycol and propylene glycol. In one specific embodiment, the present invention comprises a plasticizer, which is a mixture of triethyl citrate and glycerol.

In another embodiment the present invention provides an orally fast dissolving film formulation comprising
(a) rivaroxaban;
(b) a solubilizer selected from group consisting of sodium lauryl sulfate and polyoxyl 40 hydrogenated castor oil;
(c) a film forming polymer selected from the group consisting of polyvinyl pyrrolidone and hydroxypropyl methylcellulose;
(d) a diluent selected from group consisting of microcrystalline cellulose and maltodextrin and
(e) a plasticizer selected from group consisting of triethyl citrate and glycerol.

In a further embodiment of the invention, an orally fast dissolving film formulation of the present invention comprises about 0.5% w/w to about 20% w/w triethyl citrate, preferably about 1% w/w to about 15% w/w triethyl citrate, more preferably about 2% w/w to about 13% w/w triethyl citrate and most preferably about 3% w/w to about 10% w/w triethyl citrate based on the total weight of the film formulation.

In another embodiment of the invention, an orally fast dissolving film formulation of the present invention comprises about 1% w/w to about 40% w/w glycerol, preferably about 2% w/w to about 35% w/w glycerol, more preferably about 5% w/w to about 30% w/w glycerol, even more preferably about 6% w/w to about 25% w/w glycerol and most preferably about 10% w/w to about 20% w/w glycerol based on the total weight of the film formulation.

In another embodiment of the invention, the present invention provides an orally fast dissolving film formulation comprising
(a) about 2% w/w to about 25% w/w rivaroxaban;
(b) about 0.25% w/w to about 3% w/w sodium lauryl sulfate;
(c) about 0.01% w/w to about 1% w/w polyoxyl 40 hydrogenated castor oil;
(d) about 10% w/w to about 20% w/w polyvinyl pyrrolidone;
(e) about 25% w/w to about 40% w/w hydroxypropyl methylcellulose;
(f) about 3% w/w to about 25% w/w microcrystalline cellulose;
(g) about 3% w/w to about 10% w/w maltodextrin;
(h) about 3% w/w to about 10% w/w triethyl citrate and
(i) about 10% w/w to about 20% w/w glycerol.

In a further embodiment the present invention provides an orally fast dissolving film formulation comprising
(a) rivaroxaban;
(b) at least one solublizer selected from the group consisting of sodium lauryl sulfate and polyoxyl 40 hydrogenated castor oil;
(c) at least one film forming polymer selected from the group consisting of polyvinyl pyrrolidone, hydroxypropyl methylcellulose and pullulan;
(d) at least one diluent selected from group consisting of lactose monohydrate microcrystalline cellulose and maltodextrin;
(e) at least one plasticizer selected from group consisting of triacetin, triethyl citrate, glycerol, polyethylene glycol and propylene glycol, and
(f) at least one excipient selected from group of sweetening agents, flavouring agents and colouring agents.

In embodiments of the invention sweetening agents used in the present invention are selected from group consisting of aspartame, sucralose, dextrose, fructose, ammonium glycyrrhizinate, maltose, mannitol, sorbitol and xylitol and/or combinations thereof.

In a specific embodiment, an orally dissolving film formulation of the present invention comprises 0.5% w/w to about 20% w/w sucralose, preferably about 1% w/w to about 15% w/w sucralose, more preferably about 2% w/w to about 13% w/w sucralose and most preferably about 3% w/w to about 10% w/w sucralose based on the total weight of the film formulation.

In embodiments of the invention flavouring agents used in the present invention are selected from the group consisting of peppermint flavour, cooling flavour (menthol), flavour oils, flavouring aromatic oil, peppermint oil, clove oil, bay oil, anise oil, eucalyptus oil thyme oil, oil of bitter almonds. Flavouring agents include, vanilla, chocolate flavour, citrus oils, fruit essences, and any combinations thereof.

In embodiments of the invention colouring agents are selected from the group consisting of sunset yellow, amaranth, red iron oxide, natural juice concentrates, pigments and opacifying agents such as titanium oxide, silicon dioxide and zinc oxide, solid choco color and any combinations thereof.

In another embodiment of the invention, the present invention provides a film formulation comprising
(a) about 2% w/w to about 25% w/w rivaroxaban;
(b) about 0.25% w/w to about 3% w/w sodium lauryl sulfate;
(c) about 0.01% w/w to about 1% w/w polyoxyl 40 hydrogenated castor oil;
(d) about 10% w/w to about 20% w/w polyvinyl pyrrolidone;
(e) about 25% w/w to about 40% w/w hydroxypropyl methylcellulose;
(f) about 3% w/w to about 25% w/w microcrystalline cellulose;
(g) about 3% w/w to about 10% w/w maltodextrin;
(h) about 3% w/w to about 10% w/w triethyl citrate
(i) about 10% w/w to about 20% w/w glycerol and
(g) at least one excipient selected from the group consisting of sweetening agents, flavouring agents and colouring agents.

In more specific embodiment of the invention, the present invention provides a film formulation consisting of
(a) about 2% w/w to about 25% w/w rivaroxaban;
(b) about 0.25% w/w to about 3% w/w sodium lauryl sulfate;
(c) about 0.01% w/w to about 1% w/w polyoxyl 40 hydrogenated castor oil;
(d) about 10% w/w to about 20% w/w polyvinyl pyrrolidone;
(e) about 25% w/w to about 40% w/w hydroxypropyl methylcellulose;
(f) about 3% w/w to about 25% w/w microcrystalline cellulose;
(g) about 3% w/w to about 10% w/w maltodextrin;
(h) about 3% w/w to about 10% w/w triethyl citrate
(i) about 10% w/w to about 20% w/w glycerol and
(j) at least one excipient selected from the group consisting of sweetening agents, flavouring agents and colouring agents.

It is desirable that an orally fast dissolving film formulation of the present invention is formed into a thin film while maintaining tensile strength and toughness at desired levels.

In one embodiment, an orally fast dissolving film formulation of the present invention has a thickness of about 50 μm to about 1500 μm. The oral film formulation of the present invention has a size of about 1 cm² to about 12 cm², preferably 2 cm² to about 10 cm².

In embodiments of the invention, the film formulation of the present invention is bioequivalent to Xarelto® tablets.

In a further embodiment of the invention, the film formulation of the present invention comprising rivaroxaban is indicated; to reduce risk of stroke and systemic embolism in nonvalvular atrial fibrillation, for treatment of deep vein thrombosis (DVT), for treatment of pulmonary embolism (PE), for reduction in risk of recurrence of DVT or PE, for prophylaxis of DVT, which may lead to PE in patients undergoing knee or hip replacement surgery, for prophylaxis of venous thromboembolism (VTE) in actually ill medical patients, to reduce the risk of major cardiovascular events in patients with coronary artery disease (CAD), to reduce the risk of major thrombotic vascular events in patients with peripheral artery disease (PAD), including patients after recent lower extremity revascularization due to symptomatic PAD, for treatment of VTE and reduction in risk of recurrent VTE in pediatric patients from birth to less than 18 years and for thromboprophylaxis in pediatric patients 2 years and older with congenital heart disease after fontan procedure.

The following example is provided to illustrate the present invention. It is understood, however, that the invention is not limited to the specific conditions or details described in the example below. The example should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Example 1

### Orally Fast Dissolving Film Formulation of Rivaroxaban

| S. No. | Ingredients | mg/film | % w/w |
|---|---|---|---|
| 1 | Rivaroxaban | 20.00 | 20.00 |
| 2 | Sucralose | 6.00 | 6.00 |
| 3 | Maltodextrin | 4.50 | 4.50 |
| 4 | Polyvinyl pyrrolidone | 23.00 | 23.00 |
| 5 | Sodium lauryl sulfate | 1.00 | 1.00 |
| 6 | Iron oxide red | 0.50 | 0.50 |
| 7 | Triethyl citrate | 10.00 | 10.00 |
| 8 | Peppermint Powder | 5.00 | 5.00 |
| 9 | Hydroxypropyl methylcellulose | 30.00 | 30.00 |
| 10 | Purified Water | q.s. | NA |
| | Total | 100.00 | 100.00 |

### Process for Preparation:

1. Take required batch quantity of Purified Water in formulation vessel.
2. Add required batch quantity of polyvinyl pyrrolidone, sodium lauryl sulphate, rivaroxaban, sucralose, maltodextrin, peppermint powder and iron oxide red to the contents of steps 1 one by one and mix.
3. Transfer required batch quantity of hydroxypropyl methylcellulose base to step 2. Continue anchoring at 60±5 RPM, with homogenization for 30±5 min. Then stop anchor stirring and homogenization.
4. Add required batch quantity of triethyl citrate slowly to step 3 stir with anchor at appropriate speed.
5. Homogenize step 4 dispersion for 1 hour with anchor stirring at 50±5 RPM. During homogenization maintain the formulation dispersion temperature at 25±5° ° C. by circulating cool water through the jacket of the formulation vessel. Stop the homogenization.
6. De-aerate the step 5 dispersion for 2 hours or till slurry free from air bubbles. at pressure (600 to 735 mm) while continuing anchor stirring at 30±5 RPM. During deaeration maintain the formulation dispersion temperature at 20±5° ° C. by circulating cool water through the jacket of the formulation vessel.
7. The above slurry of step 6 is layered to form a film at a thickness of 600 to 800 μm and dried at a drying temperature 80±3° C. for 10 minutes.
8. Further the dried film is slitted and cut into film of 32×36 mm±1 mm.

### Example 2

### Orally Fast Dissolving Film Formulation of Rivaroxaban

| S. No. | Ingredients | mg/film | % w/w |
|---|---|---|---|
| 1 | Rivaroxaban | 20.00 | 18.18 |
| 2 | Sucralose | 6.00 | 5.455 |
| 3 | Lactose Monohydrate | 11.50 | 10.45 |
| 4 | Microcrystalline cellulose | 3.00 | 2.727 |
| 5 | Polyvinyl pyrrolidone | 23.00 | 20.91 |
| 6 | Sodium Lauryl Sulfate | 1.00 | 0.909 |
| 7 | Iron oxide Red | 0.50 | 0.455 |
| 8 | Triethyl citrate | 10.00 | 9.091 |
| 9 | Peppermint Powder | 5.00 | 4.545 |
| 10 | Hydroxypropyl methylcellulose | 30.00 | 27.27 |
| 11 | Purified Water | q.s. | NA |
| | Total | 110.00 | 100.00 |

### Process for Preparation:

1. Take required batch quantity of Purified Water in formulation vessel.
2. Add required batch quantity of polyvinyl pyrrolidone, sodium lauryl sulphate, rivaroxaban, sucralose, lactose monohydrate, microcrystalline cellulose, peppermint powder and iron oxide red to the contents of steps 1 one by one and mix.
3. Transfer required batch quantity of hydroxypropyl methylcellulose base to step 2. Continue anchoring at 60±5 RPM, with homogenization for 30±5 min. Then stop anchor stirring and homogenization.
4. Add required batch quantity of triethyl citrate slowly to step 3 stir with anchor at appropriate speed.
5. Homogenize step 4 dispersion for 1 hour with anchor stirring at 50±5 RPM. During homogenization maintain the formulation dispersion temperature at 25±5° C. by circulating cool water through the jacket of the formulation vessel. Stop the homogenization.
6. De-aerate the step 5 dispersion for 2 hours or till slurry free from air bubbles. at pressure (600 to 735 mm) while continuing anchor stirring at 30±5 RPM. During deaeration maintain the formulation dispersion temperature at 20±5° C. by circulating cool water through the jacket of the formulation vessel.
7. The above slurry of step 6 is layered to form a film at a thickness of 600 to 800 μm and dried at a drying temperature 80±3° C. for 10 minutes.
8. Further the dried film is slitted and cut into film of 32×36 mm±1 mm.

### Example 3

### Orally Fast Dissolving Film Formulation of Rivaroxaban

| S. No. | Ingredients | mg/film | % w/w |
|---|---|---|---|
| 1 | Rivaroxaban | 20.00 | 18.18 |
| 2 | Sucralose | 6.00 | 5.455 |
| 3 | Lactose Monohydrate | 11.50 | 10.45 |
| 4 | Microcrystalline cellulose | 3.00 | 2.727 |
| 5 | Polyvinyl pyrrolidone | 23.00 | 20.91 |
| 6 | Sodium Lauryl Sulfate | 1.00 | 0.909 |
| 7 | Polyoxyl 40 hydrogenated castor oil | 0.5 | 0.455 |
| 8 | Iron oxide Red | 0.50 | 0.455 |
| 9 | Triethyl citrate | 10.00 | 9.091 |
| 10 | Peppermint Powder | 5.00 | 4.545 |
| 11 | Hydroxypropyl methylcellulose | 14.75 | 13.41 |
| 12 | Pullulan | 14.75 | 13.41 |
| 13 | Purified Water | q.s. | NA |
| | Total | 110.00 | 100.00 |

### Process for Preparation:

1. Take required batch quantity of Purified Water in formulation vessel.
2. Add required batch quantity of polyvinyl pyrrolidone, sodium lauryl sulphate, rivaroxaban, sucralose, lactose monohydrate, microcrystalline cellulose, pullulan, peppermint powder and iron oxide red to the contents of steps 1 one by one and mix.
3. Transfer required batch quantity of hydroxypropyl methylcellulose base to step 2. Continue anchoring at 60±5 RPM, with homogenization for 30±5 min. Then stop anchor stirring and homogenization.
4. Add required batch quantity of triethyl citrate slowly to step 3 stir with anchor at appropriate speed.
5. Homogenize step 4 dispersion for 1 hour with anchor stirring at 50±5 RPM. During homogenization maintain the formulation dispersion temperature at 25±5° C. by circulating cool water through the jacket of the formulation vessel. Stop the homogenization.
6. De-aerate the step 5 dispersion for 2 hours or till slurry free from air bubbles. at pressure (600 to 735 mm) while continuing anchor stirring at 30±5 RPM. During deaeration maintain the formulation dispersion temperature at 20±5° C. by circulating cool water through the jacket of the formulation vessel.
7. The above slurry of step 6 is layered to form a film at a thickness of 600 to 800 μm and dried at a drying temperature 80±3° C. for 10 minutes.
8. Further the dried film is slitted and cut into film of 32×36 mm±1 mm.

### Example 4

### Orally Fast Dissolving Film Formulation of Rivaroxaban

| S. No. | Ingredients | mg/film | % w/w |
|---|---|---|---|
| 1 | Rivaroxaban | 20.00 | 19.05 |
| 2 | Sucralose | 6.00 | 5.71 |
| 3 | Maltodextrin | 5.00 | 4.76 |
| 4 | Polyvinyl pyrrolidone | 23.00 | 21.90 |
| 5 | Sodium lauryl sulfate | 0.25 | 0.24 |
| 6 | Iron oxide red | 0.50 | 0.48 |
| 7 | Triethyl Citrate | 15.00 | 14.29 |
| 8 | Peppermint Powder | 5.00 | 4.76 |
| 9 | Hydroxypropyl methylcellulose | 30.00 | 28.57 |
| 10 | Polyoxyl 40 hydrogenated castor oil | 0.25 | 0.24 |
| 11 | Purified Water | q.s. | NA |
| | Total | 105.00 | 100.00 |

### Process for Preparation

1. Preparation of HPMC (Hydroxypropyl methylcellulose) base: Required quantity of purified water was taken in vessel and heated. Further HPMC was added into the above vessel and stirred.
2. Slurry Preparation:
   a. Required quantity of purified water was taken into another vessel, sodium lauryl sulfate and Polyoxyl 40 hydrogenated castor oil was added into this vessel and stirred.
   b. Then rivaroxaban was added to step (a) vessel followed by polyvinyl pyrrolidone.
   c. Further iron oxide red, sucralose, maltodextrin and peppermint powder were added to the vessel contents of step (b) and stirred.
   d. Further HPMC base of step (1) was added to the contents of step (c) vessel contents and stirred
   e. Finally, triethyl citrate was added to the contents of step (d) vessel and homogenized to form the slurry.
3. Layering and Drying: The slurry of step (2) was layered to form a film thickness of 565 μm and dried at a drying temperature 70±5° C. for 10 minutes.
4. Slitting and Packing: Further the dried film is slitted, cut into film size of 32×36 mm±1 mm and packed into triple laminate pouch.

### Dissolution:

The above film was subjected to dissolution method with pH 4.5 acetate buffer containing 0.25% sodium lauryl sulfate 900 mL dissolution media in basket USP Apparatus at 100 rotations per minute and the release of rivaroxaban was analyzed by HPLC at 5, 10, 15, 20 and 30 minutes and the results are present in Table—1.

TABLE 1

| Time Point | Release of Drug (%) |
|---|---|
| 5 Min | 79 |
| 10 Min | 85 |
| 15 Min | 88 |
| 20 Min | 89 |
| 30 Min | 90 |

### EXAMPLE 5 and 6

### Orally Fast Dissolving Film Formulation of Rivaroxaban

| | | Example - 5 | | Example - 6 | |
|---|---|---|---|---|---|
| S. No. | Ingredients | mg/film | % w/w | mg/film | % w/w |
| 1 | Rivaroxaban | 20.00 | 19.05 | 20.00 | 21.74 |
| 2 | Sucralose | 6.00 | 5.71 | 6.00 | 6.52 |
| 3 | Maltodextrin | 4.75 | 4.52 | 4.75 | 5.16 |
| 4 | Polyvinyl pyrrolidone | 15.00 | 14.29 | 5.00 | 5.43 |
| 5 | Sodium lauryl sulfate | 0.50 | 0.48 | 0.50 | 0.54 |
| 6 | Iron oxide red | 0.50 | 0.48 | 0.50 | 0.54 |
| 7 | Triethyl Citrate | 5.00 | 4.76 | 5.00 | 5.43 |
| 8 | Peppermint Powder | 5.00 | 4.76 | 5.00 | 5.43 |
| 9 | HPMC | 38.00 | 36.19 | 30.00 | 32.61 |
| 10 | Polyoxy1 40 | 0.25 | 0.24 | 0.25 | 0.27 |
| | hydrogenated castor oil | | | | |
| 11 | Glycerin | 10.00 | 9.52 | 15.0 | 16.30 |
| 12 | Purified Water | q.s. | NA | q.s. | NA |
| | Total | 105.00 | 100.00 | 92.00 | 100.00 |

The process for preparation of the film is similar to the process disclosed in Example—4, with the addition of glycerin in step 2(e) of the slurry preparation.

### Example 7

### Orally Fast Dissolving Film Formulations of Rivaroxaban

| S. No | Ingredients | mg/film | % w/W |
|---|---|---|---|
| 1 | Rivaroxaban | 20.00 | 18.69% |
| 2 | Sucralose | 6.00 | 5.61% |
| 3 | Maltodextrin | 4.75 | 4.44% |
| 4 | Polyvinyl pyrrolidone | 15.00 | 14.02% |
| 5 | Sodium lauryl sulfate | 0.50 | 0.47% |
| 6 | Iron oxide red | 0.50 | 0.47% |
| 7 | Triethyl Citrate | 5.00 | 4.67% |
| 8 | Peppermint Powder | 5.00 | 4.67% |
| 9 | HPMC | 30.00 | 28.04% |
| 10 | Microcrystalline Cellulose | 5.00 | 4.67% |
| 11 | Polyoxyl 40 hydrogenated castor oil | 0.25 | 0.23% |
| 12 | Glycerin | 15.00 | 14.02% |
| 13 | Purified Water | q.s. | NA |
| | Total | 107.000 | 100.00 |

Process for preparation: The process for preparation of the film is similar to the process disclosed in Example—4, with the addition of microcrystalline cellulose in step 2(c) and glycerin in step 2(e) of slurry preparation.

### Dissolution:

The above film was subjected to dissolution method with pH 4.5 acetate buffer containing 0.25% sodium lauryl sulfate 900 mL dissolution media in basket USP Apparatus at 100 rotations per minute and the release of rivaroxaban was analyzed by HPLC at 5, 10, 15, 20 and 30 minutes and the results are present in Table—2.

TABLE 2

| Time Point | Release of Drug (%) |
|---|---|
| 5 Min | 78 |
| 10 Min | 85 |
| 15 Min | 89 |
| 20 Min | 90 |
| 30 Min | 90 |

Fasting Bioequivalence Study: An open label, balanced, randomized, two-treatment, six-sequence, three-period, single dose, crossover, oral bioavailability study of rivaroxaban orally dissolving film of above formulation in example—7 was compared with Xarelto® 20 mg tablets in seventeen normal, healthy, adult human subjects under fasting conditions. The results of fasting study of the film formulation (example—7) and reference (Xarelto® 20 mg tablets) are depicted in Table—3.

TABLE 3

| | | Cmax | AUC (0-t) | AUC (0-inf) |
|---|---|---|---|---|
| Parameter | | (ng/mL) | (h*ng/mL) | (h*ng/mL) |
| Least Square | Test | 5.3714 | 7.5903 | 7.6225 |
| mean | Reference | 5.3735 | 7.5961 | 7.6260 |
| Geometric | Test | 215.17 | 1978.88 | 2043.63 |
| LSM | Reference | 215.62 | 1990.40 | 2050.91 |

| | | | |
|---|---|---|---|
| Geometric LSM Ratio (%) | 99.79 | 99.42 | 99.65 |
| 90% confidence interval (%) | 87.13-114.30 | 92.19-107.22 | 93.15-106.59 |

### Example 8 and 9

### Orally Fast Dissolving Film Formulations of Rivaroxaban

| S. | | Example - 8 | | Example - 9 | |
|---|---|---|---|---|---|
| No. | Ingredients | mg/film | % w/w | mg/film | % w/w |
| 1 | Rivaroxaban | 20.00 | 18.69% | 20.000 | 18.69% |
| 2 | Sucralose | 6.00 | 5.61% | 6.000 | 5.61% |
| 3 | Maltodextrin | 4.75 | 4.44% | 4.750 | 4.44% |
| 4 | Polyvinyl pyrrolidone | 15.00 | 14.02% | 15.000 | 14.02% |
| 5 | Sodium lauryl sulfate | 0.58 | 0.54% | 0.580 | 0.54% |
| 6 | Iron oxide red | 0.25 | 0.23% | 0.200 | 0.19% |
| 7 | Triethyl Citrate | 5.00 | 4.67% | 5.000 | 4.67% |
| 8 | Peppermint Powder | 5.00 | 4.67% | 5.000 | 4.67% |
| 9 | HPMC | 30.00 | 28.04% | 30.000 | 28.04% |
| 10 | Microcrystalline | 5.17 | 4.83% | 5.220 | 4.88% |
| | Cellulose | | | | |
| 11 | Polyoxy1 40 | 0.25 | 0.23% | 0.250 | 0.23% |
| | hydrogenated castor | | | | |
| | oil | | | | |
| 12 | Glycerin | 15.00 | 14.02% | 15.000 | 14.02% |
| 13 | Purified Water | q.s. | NA | q.s. | NA |
| | Total | 107.00 | 100.00 | 107.00 | 100.00 |

Process for Preparation: Process for preparation is similar to the process as described in example—7 above.

### Dissolution

The above film of Example—9 was subjected to dissolution method with pH 4.5 acetate buffer containing 0.25% sodium lauryl sulfate 900 mL dissolution media in basket USP Apparatus at 100 rotations per minute and the release of rivaroxaban was analyzed by HPLC at 5, 10, 15, 20 and 30 minutes and the results are present in Table—4.

TABLE 4

| Time Point | Release of Drug (%) |
|---|---|
| 5 Min | 88 |
| 10 Min | 91 |
| 15 Min | 93 |
| 20 Min | 94 |
| 30 Min | 96 |

We claim:

1. An orally fast dissolving film formulation comprising
(a) rivaroxaban and
(b) at least one solubilizer selected from group consisting of sodium lauryl sulfate and polyoxyl 40 hydrogenated castor oil.

2. The orally fast dissolving film formulation as claimed in claim 1 comprising of about 2% w/w to about 25% w/w rivaroxaban based on the total weight of the film formulation.

3. The orally fast dissolving film formulation as claimed in claim 1 comprising solubilizer, wherein solubilizer is a mixture of sodium lauryl sulfate and polyoxyl 40 hydrogenated castor oil.

4. The orally fast dissolving film formulation as claimed in claim 3, wherein the film formulation comprises of about 0.25% w/w to about 3% w/w sodium lauryl sulfate based on the total weight of the film formulation.

5. The orally fast dissolving film formulation as claimed in claim 3, wherein the film formulation comprises of about 0.01% w/w to about 1% w/w polyoxyl 40 hydrogenated castor oil.

6. The orally fast dissolving film formulation as claimed in claim 1, wherein the film formulation further comprises
(a) a film forming polymer selected from the group consisting of polyvinyl pyrrolidone and hydroxypropyl methylcellulose;
(b) a diluent selected from group consisting of microcrystalline cellulose and maltodextrin and
(c) a plasticizer selected from group consisting of triethyl citrate and glycerol.

7. The orally fast dissolving film formulation as claimed in claim 1, wherein the film formulation further comprises at least one excipient selected from the group consisting of sweetening agents, flavouring agents and colouring agents.

8. The orally fast dissolving film formulation as claimed in claim 6, wherein the film formulation comprises
(a) film forming polymer which is a mixture of polyvinyl pyrrolidone and hydroxypropyl methylcellulose;
(b) diluent which is a mixture of microcrystalline cellulose and maltodextrin;
(c) plasticizer which is a mixture of triethyl citrate and glycerol.

9. The orally fast dissolving film formulation as claimed in claim 8, wherein the film formulation comprises
(a) about 10% w/w to about 20% w/w polyvinyl pyrrolidone;
(b) about 25% w/w to about 40% w/w hydroxypropyl methylcellulose;
(c) about 3% w/w to about 25% w/w microcrystalline cellulose;
(d) about 3% w/w to about 10% w/w maltodextrin;
(e) about 3% w/w to about 10% w/w triethyl citrate and
(f) about 10% w/w to about 20% w/w glycerol.

10. An orally fast dissolving film formulation comprising
(a) about 2% w/w to about 25% w/w rivaroxaban;
(b) about 0.25% w/w to about 3% w/w sodium lauryl sulfate;
(c) about 0.01% w/w to about 1% w/w polyoxyl 40 hydrogenated castor oil;
(d) about 10% w/w to about 20% w/w polyvinyl pyrrolidone;
(e) about 25% w/w to about 40% w/w hydroxypropyl methylcellulose;
(f) about 3% w/w to about 25% w/w microcrystalline cellulose;
(g) about 3% w/w to about 10% w/w maltodextrin;
(h) about 3% w/w to about 10% w/w triethyl citrate
(i) about 10% w/w to about 20% w/w glycerol and
(j) at least one excipient selected from the group consisting of sweetening agents, flavouring agents and colouring agents.
